(19) United States
(12) Patent Application Publication
(10) Pub. No.: US 2025/0051397 A1
(43) Pub. Date: Feb. 13, 2025

(54) VSV/NDV HYBRID VIRUSES FOR ONCOLYTIC THERAPY OF CANCER

(71) Applicant: JENNIFER ALTOMONTE, MÜNCHEN (DE)

(72) Inventors: Oliver EBERT, MÜNCHEN (DE); Jennifer ALTOMONTE, MÜNCHEN (DE)

(21) Appl. No.: 18/930,073

(22) Filed: Oct. 29, 2024

Related U.S. Application Data
(63) Continuation of application No. 17/163,468, filed on Jan. 31, 2021.
Application No. 17/163,468 is a continuation of application No. 16/097,748, filed on Oct. 30, 2018, now Pat. No. 10,906,942.

(30) Foreign Application Priority Data
May 19, 2016 (EP) 16170445.7

(51) Int. Cl. C07K 14/005; A61K 9/00; A61K 35/766; A61K 38/00; A61K 38/16; A61K 38/47; A61K 45/06; A61K 49/00; C12N 9/24; C12N 15/86

(52) U.S. Cl. CPC C07K 14/005; A61K 9/0019; A61K 9/0021; A61K 9/0085; A61K 35/766; A61K 38/162; A61K 38/47; A61K 45/06; A61K 49/00; C12N 9/2402; C12N 15/86; C12Y 302/01018; A61K 38/00; C07K 2319/50; C07K 2319/60; C12N 2760/18122; C12N 2760/20232; C12N 2760/20243; C12N 2810/6072

(57) ABSTRACT

The present invention relates to recombinant oncolytic viruses comprising a vesicular stomatitis virus (VSV), wherein the glycoprotein (G protein) of VSV is deleted; and which comprises a modified fusion protein (F protein) of Newcastle disease virus (NDV); and the hemagglutinin neuraminidase (HN) protein of NDV. The present invention further relates to nucleic acids encoding for the recombinant oncolytic virus and vectors comprising the nucleic acids. The present invention further relates to pharmaceutical compositions comprising the rVSV of the invention, the nucleic acid or the vector, further to uses as gene delivery tool and/or for tumor detection. The present invention further relates to the recombinant oncolytic vesicular stomatitis virus (VSV) for use in medicine, in particular for the diagnosis, prevention and/or treatment of cancer.

### CROSS-REFERENCE TO RELATED APPLICATIONS

This application is a continuation application of co-pending application Ser. No. 17/163,468, filed Jan. 31, 2021; which is a continuation of application Ser. No. 16/097,748, filed Oct. 30, 2018, now U.S. Pat. No. 10,906,942; which is a National Stage Application of International Application Number PCT/EP2017/062007, filed May 18, 2017; which claims priority to European Patent Application No. 16170445.7, filed May 19, 2016.

The Sequence Listing for this application is labeled “SeqList-29Oct24.xml”, which was created on Oct. 29, 2024 and is 71,592 bytes. The entire content is incorporated herein by reference in its entirety.

### FIELD OF THE INVENTION

The present invention relates to recombinant oncolytic viruses comprising a vesicular stomatitis virus (VSV), wherein the glycoprotein (G protein) of VSV is deleted; and which comprises a modified fusion protein (F protein) of Newcastle disease virus (NDV); and the hemagglutinin neuraminidase (HN) protein of NDV. The present invention further relates to nucleic acids encoding for the recombinant oncolytic virus and vectors comprising the nucleic acids. The present invention further relates to pharmaceutical compositions comprising the rVSV of the invention, the nucleic acid or the vector, further to uses as gene delivery tool and/or for tumor detection. The present invention further relates to the recombinant oncolytic vesicular stomatitis virus (VSV) for use in medicine, in particular for the diagnosis, prevention and/or treatment of cancer.

### BACKGROUND OF THE INVENTION

Oncolytic viruses (OVs) represent a novel class of therapeutic agents for cancer treatment, due to their intrinsic ability to selectively replicate and kill tumor cells, while sparing the surrounding normal tissue (Lorence et al., 1994; Coffey et al., 1998; Kirn et al., 2001; Peng et al., 2001). OV therapies involve the use of replication-competent viruses that are either inherently tumor selective or have been engineered to preferentially grow in tumor cells. During the process of malignant transformation, genetic abnormalities accumulate to provide cancer cells with growth and survival advantages. Many OVs exploit such defects in cellular signaling pathways to support their own replication in these cells. In particular, many cancer cells are impaired in their ability to secrete or respond to interferon (IFN), which is a key mechanism in the innate immune response against invading viruses in normal cells. These defects prevent tumor cells from mounting a productive antiviral defense, and, thus, replication of the OV is supported specifically in these cells.

Oncolytic viruses exert their effects both by direct killing of infected tumor cells, as well as indirect effects, such as destruction of tumor vasculature and induction of adaptive immune responses, which can be directed against the tumor and lead to destruction of neighboring uninfected tumor cells. Furthermore, genetics systems are available, which allow us to engineer and rescue recombinant viral vectors from plasmid DNA. In this way, viruses can be modified to increase tumor specificity or to express therapeutic genes and/or reporter genes.

Over the last decade, significant progress has been made in the development of enhanced OV therapies, and a variety of vectors have entered clinical trials (Kirn et al., 2001; Everts and van der Poel, 2005; Patel and Kratzke, 2013). Recently, a recombinant herpes simplex virus I vector was the first oncolytic virus to be approved by the FDA for use as a clinical agent (press release Oct. 27, 2015, Amgen), and approval in Europe is expected to follow. However, in general, clinical trial results are often disappointing due to a lack of reliable and predictive preclinical models and due to inadequate tumor responses to most OV therapies in immune competent hosts.

Therapeutic efficacy of oncolytic viral therapy often comes as a trade-off with safety, such that potent vectors are often associated with toxicity, while safer viruses provide attenuated therapeutic effects. Despite promising preclinical data, the development of vesicular stomatitis virus (VSV) as a clinical agent has been substantially hampered by the fact that severe neurotoxicity has been observed in rodents and nonhuman primates in response to treatment with wild-type VSV (van den Pool et al., 2002; Johnson et al., 2007). In addition to the safety aspect, the rapid accumulation of high intratumoral titers of VSV, as a consequence of its short life cycle, results in an early and potent innate immune response, which severely limits the ability of the virus to efficiently spread and destroy the entire tumor mass before being cleared from the host (Altomonte et al., 2008).

Newcastle disease virus (NDV) has been shown to be a potent oncolytic agent with an attractive safety profile in humans; however, the use of NDV poses an environmental risk to birds and the poultry industry, as avian species are the natural hosts of the virus. Although mesogenic and velogenic strains of NDV have been shown to be the most effective as oncolytic viruses, they have been classified by the USDA as select agents since 2008, prohibiting their use and thereby severely impeding the development of NDV into a clinical agent (www.selectagents.gov).

To improve the safety of oncolytic VSV vectors, researchers have investigated a variety of approaches. First, recombinant VSVs harboring nucleotide substitutions or deletions to alter the amino acid composition of the matrix (M) protein at position 51 interfere with the ability of the endogenous M protein to inhibit cellular transcription and nucleocytoplasmic RNA transfer, allowing for antiviral cellular responses to be launched. Although these vectors have been shown to be safer than wildtype, intratumoral replication is also attenuated (Stojdl et al., 2003; Ebert et al., 2005), limiting the therapeutic value of this approach. Another strategy to improve the safety of VSV involves the incorporation of miRNA target sequences into the virus genome in order to modify the tropism of the virus, however these vectors are also less effective (Edge et al., 2008; Kelly et al., 2010).

Various attempts are being explored to engineer the NDV genome to limit the pathogenicity in avian species, see e.g. patent application WO 2015/032755 A1. Whether or not these modifications will truly improve safety and the effect of these modifications on the oncolytic capacity of the vectors remain to be seen.

Thus, there is a need in the art for improved means and methods for oncolytic virotherapy as well as for improved oncolytic viruses.

### BRIEF SUMMARY OF THE INVENTION

According to the present invention this object is solved by a recombinant oncolytic virus, comprising a vesicular stomatitis virus (VSV),
wherein the glycoprotein (G protein) of VSV is deleted, and which comprises
   a modified fusion protein (F protein) of Newcastle disease virus (NDV); and
   the hemagglutinin neuraminidase (HN) protein of NDV.

According to the present invention this object is solved by a nucleic acid encoding the recombinant oncolytic virus of the invention.

According to the present invention this object is solved by a vector comprising the nucleic acid of the invention.

According to the present invention this object is solved by a pharmaceutical composition, comprising:
(i) the recombinant oncolytic virus, the nucleic acid or the vector of the present invention; and
(ii) optionally, pharmaceutically acceptable carrier(s) and/or excipient(s).

According to the present invention this object is solved by the use of the recombinant oncolytic virus, the nucleic acid or the vector of the present invention or the pharmaceutical composition of the present invention, as gene delivery tool, and/or (noninvasive) imaging of virus biodistribution, and/or for tumor detection.

According to the present invention this object is solved by providing the recombinant oncolytic virus, the nucleic acid or the vector of the present invention or the pharmaceutical composition of the present invention for use in medicine.

According to the present invention this object is solved by providing the recombinant oncolytic virus, the nucleic acid or the vector of the present invention or the pharmaceutical composition of the present invention for use in the diagnosis, prevention and/or treatment of cancer.

According to the present invention this object is solved by a method of diagnosis, prevention and/or treatment of cancer comprising the step of administering to a subject in need thereof a therapeutically effective amount of the recombinant oncolytic virus, the nucleic acid or the vector of the present invention or the pharmaceutical composition of the present invention.

### DETAILED DESCRIPTION OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “20 to 100 nucleotides” should be interpreted to include not only the explicitly recited values of 20 to 100, but also include individual values and sub-ranges within the indicated range. Thus, included in this numerical range are individual values such as 20, 21, 22, 23, 24, 25, 26, 27, 28, 29, . . . 97, 98, 100 and sub-ranges such as from 25 to 35, from 20 to 40, from 25 to 50, etc. This same principle applies to ranges reciting only one numerical value, such as “at least 25 nucleotides”. Furthermore, such an interpretation should apply regardless of the breadth of the range or the characteristics being described.

Oncolytic viruses and VSV vectors

As discussed above, the present invention provides recombinant oncolytic viruses.

In particular, the present invention provides recombinant oncolytic VSV viruses wherein the glycoprotein protein of VSV is pseudotyped.

Among the most promising OV vector platforms under development are vesicular stomatitis virus (VSV) and Newcastle disease virus (NDV).

Vesicular stomatitis virus (VSV) is a negative-strand RNA virus of the Rhabdovirus family.

VSV vectors are very attractive oncolytic agents due to their inherent tumor specificity and rapid replication cycle, which results in high intratumoral titers and subsequent tumor cell lysis.

The genome of VSV is a single molecule of negative-sense RNA that encodes five major proteins: glycoprotein (G), large polymerase protein (L), phosphoprotein (P), matrix protein (M) and nucleoprotein (N). The total genome is about 11,000 nucleotides.

The VSV G protein enables viral entry. It mediates viral attachment to an LDL receptor (LDLR) or an LDLR family member present on the host cell. Following binding the VSV-LDLR complex is rapidly endocytosed. It then mediates fusion of the viral envelope with the endosomal membrane. VSV enters the cell through partially clathrin-coated vesicles; virus-containing vesicles contain more clathrin and clathrin adaptor than conventional vesicles. Virus-containing vesicles recruit components of the actin machinery for their interaction, thus inducing its own uptake. Replication occurs in the cytoplasm.

The VSV L protein is encoded by half the genome, and combines with the phosphoprotein to catalyze replication of the mRNA.

The VSV M protein is encoded by an mRNA that is 831 nucleotides long and translates to a 229 amino acid-protein. The predicted M protein sequence does not contain any long hydrophobic or nonpolar domains that might promote membrane association. The protein is rich in basic amino acids and contains a highly basic amino terminal domain.

VSV Indiana complete genome SEQ ID NO: 1
NCBI GenBank accession No. J02428.1
VSV Indiana G protein SEQ ID NOs: 2 and 3
See GenBank accession No. X03633.1 for nucleotide and amino acid sequence.

Newcastle disease virus (NDV) is an avian virus of the Paramyxovirus family. Members of this family have a single stranded linear RNA. The total genome is about 16,000 nucleotides. Replication of the virus takes place in the cytoplasm of the host cell.

It is similar to VSV in that it is a negative-stand RNA virus and has been developed as an oncolytic virus, due to its innate ability to replicate and cause lysis in tumor cells, while leaving healthy cells unharmed (Altomonte et al., 2010; Vigil et al., 2007). Phase I-II clinical trials have shown promise for NDV and suggest that there is minimal toxicity related to the therapy. A major benefit of NDV as an oncolytic agent is that the viral envelope, which is comprised of a hemagglutinin-neuraminidase (HN) and fusion (F) protein, mediates not only virus attachment and fusion to the target cell, but it causes fusion of infected cells to their neighboring uninfected cells, providing a potent mechanism for viral spread and tumor cell killing. Furthermore, new evidence indicates that the syncytia formation caused by cell-cell fusion results in a multimodal cell death response, which can synergize with the direct oncolytic effect of the virus for a potent mechanism of tumor destruction (Cuadrado-Castano et al., 2015).

Two proteins of Newcastle disease virus are inserted in the envelope. They are the hemagglutinin/neuraminidase protein (HN) and the fusion protein (F). These two proteins are important in determining the virulence of the virus and how the virus infects host cells.

The hemagglutinin/neuraminidase protein has two sections that are of interest: (1) The hemagglutinin section, which is an attachment protein and binds to receptors on the outside of the membrane of host cells including red blood cells. (2) The neuraminidase section is the active site of an enzyme that aids in the release of the virus from the membrane of host cells. The activity of this enzyme affects the time taken for the virus to elute from red blood cells.

The fusion protein F fuses the virus envelope to the membrane of the host cell. This allows penetration of the host cell by the viral genome. In order for fusion to occur, the shape of the native fusion protein must be changed. This change happens when a host cell protease cleaves the protein at a specific cleavage site. After this has happened, the fusion protein is activated and can now fuse to the membrane of the cell. The sequence of the amino acids around the cleavage site determines the range of proteases that can activate cleavage of the protein. This sequence therefore determines the virulence.

NDV F protein is responsible for viral fusion with the cell membrane and for viral spread from cell to cell via formation of syncytia. The presence of a multibasic cleavage site within the F protein allows for protein cleavage and activation by a broad range of proteases and is a determinant of virulence in velogenic viral strains.

To increase oncolytic potency of a highly attenuated lentogenic Hitchner B1 NDV strain, a polybasic cleavage site was introduced into the F protein to generate rNDV/F3aa (Vigil et al., 2007). While the resultant virus exhibited only an intermediate virulence phenotype based on a mean death time in embryonated eggs, the virus formed large syncytia and was enhanced in its replication in cancer cells, leading to enhanced oncolytic effects in various animal tumor models. Similar findings were shown when the F protein of the lentogenic NDV La Sota strain was modified in an analogous fashion (Peeters et al., 1999). The inventors have further demonstrated that a single amino acid substitution from leucine to alanine at amino acid 289 (L289A) in the F3aa-modified fusion protein results in substantially greater syncytial formation and tumor necrosis than the virus bearing only the F3aa mutation, without any additional toxicity (Altomonte et al., 2010).

The fusogenic and oncolytic activity of the rNDV/F3aa strain can be further enhanced by a point mutation in the F protein at residue 289 from leucine to alanine, generating rNDV/F3aa (L289A). In an orthotopic immunocompetent liver tumor rat model, administration of the mutant virus via hepatic arterial infusion resulted in significant syncytia formation and necrosis, which translated to a significant 20% prolongation of survival over treatment with the original rNDV/F3aa virus (Altomonte et al., 2010).

NDV Hitchner B1 complete genome SEQ ID NO: 4
GenBank accession No. AF375823
NDV HN protein SEQ ID NOs: 5 and 6
See GenBank accession No. AF375823 and NCBI Gene ID 912270 for nucleic acid and amino acid sequence.
NDV F protein SEQ ID NOs: 7 and 8
See GenBank accession No. AF375823 and NCBI Gene ID 912271 for nucleic acid and amino acid sequence.

| SEQ ID NO: 8 | |
|---|---|
| MGSRPFTKNP APMMLTIRVA LVLSCICPAN SIDGRPFAAA GIVVTGDKAV NIYTSSQTGS | 60 |
| IIVKLLPNLP KDKEACAKAP LDAYNRTLTT LLTPLGDSIR RIQESVTTSG GGRQGRLIGA | 120 |
| IIGGVALGVA TAAQITAAAA LIQAKQNAAN ILRLKESIAA TNEAVHEVTD GLSQLAVAVG | 180 |
| KMQQFVNDQF NKTAQELDCI KIAQQVGVEL NLYLTELTTV FGPQITSPAL NKLTIQALYN | 240 |
| LAGGNMDYLL TKLGIGNNQL SSLIGSGLIT GNPILYDSQT QLLGIQVTLP SVGNLNNMRA | 300 |
| TYLETLSVST TRGFASALVP KVVTQVGSVI EELDTSYCIE TDLDLYCTRI VTFPMSPGIY | 360 |
| SCLSGNTSAC MYSKTEGALT TPYMTIKGSV IANCKMTTCR CVNPPGIISQ NYGEAVSLID | 420 |
| KQSCNVLSLG GITLRLSGEF DVTYQKNISI QDSQVIITGN LDISTELGNV NNSISNALNK | 480 |
| LEESNRKLDK VNVKLTSTSA LITYIVLTII SLVFGILSLI LACYLMYKQK AQQKTLLWLG | 540 |
| NNTLDQMRAT TKM | 553 |

NDV F3aa-modified fusion protein SEQ ID NOs: 9 and 10

| (Park et al., 2006 and Altomonte et al., 2010) | |
|---|---|
| SEQ ID NO: 9 | |
| ATGGGCTCCAGACCTTCTACCAAGAACCCAGCACCTATGATGCTGACTATCCGGGTC | |
| GCGCTGGTACTGAGTTGCATCTGCCCGGCAAACTCCATTGATGGCAGGCCTCTTGCA | |
| GCTGCAGGAATTGTGGTTACAGGAGACAAAGCAGTCAACATATACACCTCATCCCA | |
| GACAGGATCAATCATAGTTAAGCTCCTCCCGAATCTGCCCAAGGATAAGGAGGCAT | |
| GTGCGAAAGCCCCCTTGGATGCATACAACAGGACATTGACCACTTTGCTCACCCCCC | |
| TTGGTGACTCTATCCGTAGGATACAAGAGTCTGTGACTACATCTGGAGGGCGGAGA | |
| CAGAGGCGCTTTATAGGCGCCATTATTGGCGGTGTGGCTCTTGGGGTTGCAACTGCC | |
| GCACAAATAACAGCGGCCGCAGCTCTGATACAAGCCAAACAAAATGCTGCCAACAT | |
| CCTCCGACTTAAAGAGAGCATTGCCGCAACCAATGAGGCTGTGCATGAGGTCACTG | |
| ACGGATTATCGCAACTAGCAGTGGCAGTTGGGAAGATGCAGCAGTTTGTTAATGAC | |
| CAATTTAATAAAACAGCTCAGGAATTAGACTGCATCAAAATTGCACAGCAAGTTGG | |
| TGTAGAGCTCAACCTGTACCTAACCGAATTGACTACAGTATTCGGACCACAAATCAC | |
| TTCACCTGCCTTAAACAAGCTGACTATTCAGGCACTTTACAATCTAGCTGGTGGGAA | |
| TATGGATTACTTATTGACTAAGTTAGGTATAGGGAACAATCAACTCAGCTCATTAAT | |
| CGGTAGCGGCTTAATCACCGGTAACCCTATTCTATACGACTCACAGACTCAACTCTT | |
| GGGTATACAGGTAACTCTACCTTCAGTCGGGAACCTAAATAATATGCGTGCCACCTA | |
| CTTGGAAACCTTATCCGTAAGCACAACCAGGGGATTTGCCTCGGCACTTGTCCCAAA | |
| AGTGGTGACACAGGTCGGTTCTGTGATAGAAGAACTTGACACCTCATACTGTATAGA | |
| AACTGACTTAGATTTATATTGTACAAGAATAGTAACGTTCCCTATGTCCCCTGGTATT | |
| TACTCCTGCTTGAGCGGCAATACATCGGCCTGTATGTACTCAAAGACCGAAGGCGCA | |
| CTTACTACACCATATATGACTATCAAAGGCTCAGTCATCGCTAACTGCAAGATGACA | |
| ACATGTAGATGTGTAAACCCCCCGGGTATCATATCGCAAAACTATGGAGAAGCCGT | |
| GTCTCTAATAGATAAACAATCATGCAATGTTTTATCCTTAGGCGGGATAACTTTAAG | |
| GCTCAGTGGGGAATTCGATGTAACTTATCAGAAGAATATCTCAATACAAGATTCTCA | |
| AGTAATAATAACAGGCAATCTTGATATCTCAACTGAGCTTGGGAATGTCAACAACTC | |
| GATCAGTAATGCTTTGAATAAGTTAGAGGAAAGCAACAGAAAACTAGACAAAGTCA | |
| ATGTCAAACTGACCAGCACATCTGCTCTCATTACCTATATCGTTTTGACTATCATATC | |
| TCTTGTTTTTGGTATACTTAGCCTGATTCTAGCATGCTACCTAATGTACAAGCAAAAG | |
| GCGCAACAAAAGACCTTATTATGGCTTGGGAATAATACCCTAGATCAGATGAGAGC | |
| CACTACAAAAATGTGA | |
| (Park et al., 2006 and Altomonte et al., 2010) | |
| SEQ ID NO: 10 | |
| MGSRPFTKNP APMMLTIRVA LVLSCICPAN SIDGRPFAAA GIVVTGDKAV NIYTSSQTGS | 60 |
| IIVKLLPNLP KDKEACAKAP LDAYNRTLTT LLTPLGDSIR RIQESVTTSG GRRQRRFIGA | 120 |
| IIGGVALGVA TAAQITAAAA LIQAKQNAAN ILRLKESIAA TNEAVHEVTD GLSQLAVAVG | 180 |
| KMQQFVNDQF NKTAQELDCI KIAQQVGVEL NLYLTELTTV FGPQITSPAL NKLTIQALYN | 240 |
| LAGGNMDYLL TKLGIGNNQL SSLIGSGLIT GNPILYDSQT QLLGIQVTLP SVGNLNNMRA | 300 |
| TYLETLSVST TRGFASALVP KVVTQVGSVI EELDTSYCIE TDLDLYCTRI VTFPMSPGIY | 360 |
| SCLSGNTSAC MYSKTEGALT TPYMTIKGSV IANCKMTTCR CVNPPGIISQ NYGEAVSLID | 420 |
| KQSCNVLSLG GITLRLSGEF DVTYQKNISI QDSQVIITGN LDISTELGNV NNSISNALNK | 480 |
| LEESNRKLDK VNVKLTSTSA LITYIVLTII SLVFGILSLI LACYLMYKQK AQQKTLLWLG | 540 |
| NNTLDQMRAT TKM | |

NDV F3aa-modified fusion protein with L289A SEQ ID NOs: 11 and 12

| (See also Altomonte et al., 2010) | |
|---|---|
| SEQ ID NO: 11 | |
| ATGGGCTCCAGACCTTCTACCAAGAACCCAGCACCTATGATGCTGACTATCCGGGTC | |
| GCGCTGGTACTGAGTTGCATCTGCCCGGCAAACTCCATTGATGGCAGGCCTCTTGCA | |
| GCTGCAGGAATTGTGGTTACAGGAGACAAAGCAGTCAACATATACACCTCATCCCA | |
| GACAGGATCAATCATAGTTAAGCTCCTCCCGAATCTGCCCAAGGATAAGGAGGCAT | |
| GTGCGAAAGCCCCCTTGGATGCATACAACAGGACATTGACCACTTTGCTCACCCCCC | |
| TTGGTGACTCTATCCGTAGGATACAAGAGTCTGTGACTACATCTGGAGGGCGGAGA | |
| CAGAGGCGCTTTATAGGCGCCATTATTGGCGGTGTGGCTCTTGGGGTTGCAACTGCC | |
| GCACAAATAACAGCGGCCGCAGCTCTGATACAAGCCAAACAAAATGCTGCCAACAT | |
| CCTCCGACTTAAAGAGAGCATTGCCGCAACCAATGAGGCTGTGCATGAGGTCACTG | |
| ACGGATTATCGCAACTAGCAGTGGCAGTTGGGAAGATGCAGCAGTTTGTTAATGAC | |
| CAATTTAATAAAACAGCTCAGGAATTAGACTGCATCAAAATTGCACAGCAAGTTGG | |
| TGTAGAGCTCAACCTGTACCTAACCGAATTGACTACAGTATTCGGACCACAAATCAC | |
| TTCACCTGCCTTAAACAAGCTGACTATTCAGGCACTTTACAATCTAGCTGGTGGGAA | |
| TATGGATTACTTATTGACTAAGTTAGGTATAGGGAACAATCAACTCAGCTCATTAAT | |
| CGGTAGCGGCTTAATCACCGGTAACCCTATTCTATACGACTCACAGACTCAACTCTT | |
| GGGTATACAGGTAACTGCACCTTCAGTCGGGAACCTAAATAATATGCGTGCCACCT | |
| ACTTGGAAACCTTATCCGTAAGCACAACCAGGGGATTTGCCTCGGCACTTGTCCCAA | |
| AAGTGGTGACACAGGTCGGTTCTGTGATAGAAGAACTTGACACCTCATACTGTATAG | |
| AAACTGACTTAGATTTATATTGTACAAGAATAGTAACGTTCCCTATGTCCCCTGGTA | |
| TTTACTCCTGCTTGAGCGGCAATACATCGGCCTGTATGTACTCAAAGACCGAAGGCG | |
| CACTTACTACACCATATATGACTATCAAAGGCTCAGTCATCGCTAACTGCAAGATGA | |
| CAACATGTAGATGTGTAAACCCCCCGGGTATCATATCGCAAAACTATGGAGAAGCC | |
| GTGTCTCTAATAGATAAACAATCATGCAATGTTTTATCCTTAGGCGGGATAACTTTA | |
| AGGCTCAGTGGGGAATTCGATGTAACTTATCAGAAGAATATCTCAATACAAGATTCT | |
| CAAGTAATAATAACAGGCAATCTTGATATCTCAACTGAGCTTGGGAATGTCAACAAC | |
| TCGATCAGTAATGCTTTGAATAAGTTAGAGGAAAGCAACAGAAAACTAGACAAAGT | |
| CAATGTCAAACTGACCAGCACATCTGCTCTCATTACCTATATCGTTTTGACTATCATA | |
| TCTCTTGTTTTTGGTATACTTAGCCTGATTCTAGCATGCTACCTAATGTACAAGCAAA | |
| AGGCGCAACAAAAGACCTTATTATGGCTTGGGAATAATACCCTAGATCAGATGAGA | |
| GCCACTACAAAAATGTGA | |
| (See also Altomonte et al., 2010) | |
| SEQ ID NO: 12 | |
| MGSRPFTKNP APMMLTIRVA LVLSCICPAN SIDGRPFAAA GIVVTGDKAV NIYTSSQTGS | 60 |
| IIVKLLPNLP KDKEACAKAP LDAYNRTLTT LLTPLGDSIR RIQESVTTSG GRRQRRFIGA | 120 |
| IIGGVALGVA TAAQITAAAA LIQAKQNAAN ILRLKESIAA TNEAVHEVTD GLSQLAVAVG | 180 |
| KMQQFVNDQF NKTAQELDCI KIAQQVGVEL NLYLTELTTV FGPQITSPAL NKLTIQALYN | 240 |
| LAGGNMDYLL TKLGIGNNQL SSLIGSGLIT GNPILYDSQT QLLGIQVTAP SVGNLNNMRA | 300 |
| TYLETLSVST TRGFASALVP KVVTQVGSVI EELDTSYCIE TDLDLYCTRI VTFPMSPGIY | 360 |
| SCLSGNTSAC MYSKTEGALT TPYMTIKGSV IANCKMTTCR CVNPPGIISQ NYGEAVSLID | 420 |
| KQSCNVLSLG GITLRLSGEF DVTYQKNISI QDSQVIITGN LDISTELGNV NNSISNALNK | 480 |
| LEESNRKLDK VNVKLTSTSA LITYIVLTII SLVFGILSLI LACYLMYKQK AQQKTLLWLG | 540 |
| NNTLDQMRAT TKM | 553 |

As discussed above, the present invention provides recombinant oncolytic VSV viruses, wherein the glycoprotein protein of VSV is pseudotyped.

Recently, the concept of exchanging the glycoprotein (“pseudotyping”) of a virus with that of a heterologous virus has been demonstrated as an effective means of altering virus tropism. Using this approach, the viral backbone is kept intact, and therefore, it is hypothesized that virus replication in susceptible cells should be minimally effected. One group has described a VSV vector that has been pseudotyped with the envelope protein of the lymphocytic choriomemingitis virus (LCMV-GP), which has been shown to be significantly less neurotropic than the wildtype vector (Muik et al., 2011). Similarly, the VSV glycoprotein has been exchanged with that of measles virus and modified with single-chain variable antibody fragments to retarget VSV to cancer cells expressing discrete surface receptors (Ayala-Breton et al., 2012).

In the present invention a recombinant oncolytic virus is provided,
comprising a vesicular stomatitis virus (VSV),
wherein the glycoprotein (G protein) of VSV is deleted, and which comprises
a modified fusion protein (F protein) of Newcastle disease virus (NDV); and
the hemagglutinin neuraminidase (HN) protein of NDV.

In a preferred embodiment, the modified fusion protein (F protein) of NDV is the F3aa-modified F protein, and/or comprises at least one amino acid substitution in the protease cleavage site, preferably in position L289, e.g. L289A.

In a preferred embodiment, the G protein of VSV is replaced by the modified fusion protein and HN protein of NDV.

The recombinant oncolytic virus furthermore comprises the remaining proteins of VSV, namely the large polymerase protein (L), phosphoprotein (P), matrix protein (M) and nucleoprotein (N).

For example, the endogenous glycoprotein of VSV can be deleted from a plasmid encoding the full-length VSV genome. The NDV glycoprotein, comprising a modified fusion protein (NDV/F (L289A)) and hemagglutinin-neuraminidase (NDV/HN), can be inserted as discrete transcription units between the VSV matrix (M) and large polymerase (L) genes. See e.g. FIG. 1.

In an embodiment of the rVSV (vector) of the present invention, the modified fusion protein (F protein) of NDV comprises or consists of the amino acid sequence of SEQ ID NO: 10 [=aa sequence of F3aa protein] or SEQ ID NO: 12 [=aa sequence of F3aa protein/L289A], or
an amino acid sequence having at least 60%, or preferably at least 70% or 80% or 90% or 95% sequence identity to the amino acid sequence of SEQ ID NOs: 10 or 12, and/or
wherein the modified fusion protein (F protein) of NDV is encoded by a nucleotide sequence of SEQ ID NO. 9 [=nucleotide sequence of F3aa protein] or SEQ ID NO: 11/=nucleotide sequence of F3aa protein/L289A], or
a nucleotide sequence having at least 60%, or preferably at least 70% or 80% or 90% or 95% sequence identity to the nucleotide sequence of SEQ ID NOs: 9 or 11.

In an embodiment of the rVSV (vector) of the present invention, wherein the HN protein of NDV comprises or consists of the amino acid sequence of SEQ ID NO: 6, or
an amino acid sequence having at least 60%, or preferably at least 70% or 80% or 90% or 95% sequence identity to the amino acid sequence of SEQ ID NO: 6, and/or
wherein the HN protein of NDV is encoded by a nucleotide sequence of SEQ ID NO: 5 or a nucleotide sequence having at least 60%, or preferably at least 70% or 80% or 90% or 95% sequence identity to the nucleotide sequence of SEQ ID NO: 5.

As discussed above, the present invention comprises nucleic acids encoding the oncolytic viruses of the present invention.

As discussed above, the present invention comprises vectors comprising the nucleic acids of the present invention.

In preferred embodiments, the vector of the present invention further comprises:
reporter gene(s),
   such as HSV1-sr39TK, the sodium iodide symporter (NIS), somatostatin receptor 2 (SSTR2), luciferase (Firefly or Renilla), green fluorescence protein (GFP), lacZ; tyrosinase
gene(s) to be delivered to target cell(s) or tissue,
   such as gene(s) to be delivered to tumor cell(s) or tumor(s),
   e.g.
      immune stimulating genes, such as IFN-α, IFN-β, or granulocyte macrophage colony-stimulating factor (GM-CSF);
      immune checkpoint inhibitory antibodies, such as PD-1, PD1-L, CTLA-4, LAG-3, or B7-H3; and/or
      tumor associated antigens (TAA) for vaccination (specific for the tumor being targeted);
or combinations thereof.

In an embodiment, the nucleic acid or the vector of the present invention comprises or consists of the nucleotide sequence of SEQ ID NO: 13[=nucleotide sequence of complete virus/vector construct], or
a nucleotide sequence having at least 60%, or preferably at least 70% or 80% or 90% or 95% sequence identity to the nucleotide sequence of SEQ ID NO: 13, and/or
comprises or consists of the nucleotide sequence coding for an amino acid sequence with SEQ ID NOs: 6, 12, 14 to 17[=aa sequence of the proteins encoded by the virus/vector construct], or
a nucleotide sequence having at least 60%, or preferably at least 70% or 80% or 90% or 95% sequence identity to the nucleotide sequence coding for an amino acid sequence with SEQ ID NOs: 6, 12, 14 to 17.
SEQ ID NO: 13 shows the nucleotide sequence of complete virus/vector construct.
SEQ ID NOs: 14-17 and 12 and 6 show the amino acid sequences of the proteins encoded by SEQ ID NO: 13, namely:
SEQ ID NO: 14 amino acid sequence of the protein VSV-N;
SEQ ID NO: 15 amino acid sequence of the protein VSV-P;
SEQ ID NO: 16 amino acid sequence of the protein VSV-M;
SEQ ID NO: 12 amino acid sequence of the protein NDV-F3aa (L289A);
SEQ ID NO: 6 amino acid sequence of the protein NDV-HN;
SEQ ID NO: 17 amino acid sequence of the protein VSV-L.

### Pharmaceutical Compositions

As discussed above, the present invention provides a pharmaceutical composition, comprising:
(i) the recombinant oncolytic virus of the present invention or a nucleic acid of the present invention or a vector of the present invention; and
(ii) optionally, pharmaceutically acceptable carrier(s) and/or excipient(s).

In one embodiment, the pharmaceutical composition comprises further drug(s), such as
chemotherapeutic agent(s),
radiotherapeutic agent(s),
tumor vaccine(s),
immune checkpoint inhibitor(s),
cell carrier system(s),
small molecule inhibitor(s),
embolization agent(s),
shielding polymer(s).

In one embodiment, the pharmaceutical composition is formulated for systemic delivery, tumor injection, intravenous administration, intra-arterial administration, and/or for intradermal, subcutaneous, intramuscular, intravenous, intraosseous, intraperitoneal, intrathecal, epidural, intracardiac, intraarticular, intracavernous, intracerebral, intracerebroventricular and intravitreal injection(s).

Uses as Gene Delivery Tool and/or for Tumor Detection

As discussed above, the present invention provides the use of the recombinant oncolytic virus, the nucleic acid or the vector of the present invention or the pharmaceutical composition of the present invention:
as gene delivery tool
   and/or
(noninvasive) imaging of virus biodistribution
   and/or
for tumor detection.

In an embodiment, the vectors of the present invention comprise gene(s) to be delivered to target cell(s) or tissue,
such as gene(s) to be delivered to tumor cell(s) or tumor(s),
e.g.
   immune stimulating genes, such as IFN-α, IFN-β, or granulocyte macrophage colony-stimulating factor (GM-CSF);
   immune checkpoint inhibitory antibodies, such as PD-1, PD1-L, CTLA-4, LAG-3, or B7-H3; and/or
   tumor associated antigens (TAA) for vaccination (specific for the tumor being targeted).

In an embodiment, the vectors of the present invention comprise reporter gene(s), such as HSV1-sr39TK, the sodium iodide symporter (NIS), somatostatin receptor 2 (SSTR2), luciferase (Firefly or Renilla), green fluorescence protein (GFP), lacZ, tyrosinase and are then suitable for e.g. noninvasive imaging of virus biodistribution or tumor detection.

### Medical Uses

As discussed above, the present invention provides the recombinant oncolytic viruses, the nucleic acids or the vectors of the present invention or the pharmaceutical composition of the present invention for use in medicine.

As discussed above, the present invention provides the recombinant oncolytic viruses, the nucleic acids or the vectors of the present invention or the pharmaceutical composition of the present invention for use in the diagnosis, prevention and/or treatment of cancer.

In one embodiment, the present invention provides the recombinant oncolytic viruses, the nucleic acids or the vectors of the present invention or the pharmaceutical composition of the present invention for use in oncolytic therapy.

The term “oncolytic virotherapy” as used herein refers to therapy of cancer by administration of oncolytic viruses, nucleic acids encoding them or respective vectors to induce tumor regression.

In one embodiment, the recombinant oncolytic viruses, the nucleic acids or the vectors of the present invention or the pharmaceutical composition of the present invention are provided for use in combination with other therapies.

Said other therapies can be:
cell carrier systems,
   e.g. T cells, dendritic cells, NK cells, mesenchymal stem cells, immunotherapies,
   e.g. tumor vaccines or immune checkpoint inhibitors,
and/or
standard tumor therapies,
   e.g. radiofrequency ablation, chemotherapy, embolization, small molecule inhibitors.

In one embodiment, the administration is systemic, intravenous, intra-arterial, via injection into tumor, and/or via intradermal, subcutaneous, intramuscular, intravenous, intraosseous, intraperitoneal, intrathecal, epidural, intracardiac, intraarticular, intracavernous, intracerebral, intracerebroventricular and intravitreal injection(s).

Methods of Diagnosis, Prevention and/or Treatment of Cancer

As discussed above, the present invention provides a method of diagnosis, prevention and/or treatment of cancer comprising the step of
administering to a subject in need thereof a therapeutically effective amount of the recombinant oncolytic virus, the nucleic acid or the vector of the present invention or the pharmaceutical composition of the present invention.

A therapeutically effective amount of a recombinant oncolytic virus, nucleic acid or vector of the present invention is the amount which results in the desired therapeutic result, in particular tumor regression.

The recombinant viruses, nucleic acids, vectors or their pharmaceutical composition(s) are preferably administered in multiple cycles over a period of time, such as for several days up to several weeks.

In one embodiment, the administration is systemic, intravenous, intra-arterial, via injection into tumor, and/or via intradermal, subcutaneous, intramuscular, intravenous, intraosseous, intraperitoneal, intrathecal, epidural, intracardiac, intraarticular, intracavernous, intracerebral, intracerebroventricular and intravitreal injection(s).

In one embodiment, the recombinant oncolytic virus, nucleic acid or vector of the present invention or the pharmaceutical composition of the present invention are provided are administered to a subject in need thereof in combination with other therapies.

Said other therapies can be:
cell carrier systems,
   e.g. T cells, dendritic cells, NK cells, mesenchymal stem cells, immunotherapies,
   e.g. tumor vaccines or immune checkpoint inhibitors,
and/or
standard tumor therapies,
   e.g. radiofrequency ablation, chemotherapy, embolization, small molecule inhibitors,

### Further Description of Preferred Embodiments

The invention discloses a pseudotyped VSV vector, in which the endogenous glycoprotein has been deleted and exchanged with modified envelope proteins of Newcastle disease virus (NDV).

It has previously been demonstrated that a modification of the fusion protein of the Hitchner B1 strain of NDV by introduction of a polybasic protease cleavage site (rNDV/F3aa), allows for efficient syncytia formation in a wide range of cells in the absence of exogenous proteases (Vigil et al., 2007). We have further demonstrated that a single amino acid substitution from leucine to alanine at amino acid 289 (L289A) in the F3aa-modified fusion protein results in substantially greater syncytial formation and tumor necrosis than the virus bearing only the F3aa mutation, without any additional toxicity (Altomonte et al., 2010).

According to the present invention, said modified hyperfusogenic F protein has been inserted, together with the NDV HN attachment protein, into the VSV G-deleted vector.

By creating a hybrid of these two potent oncolytic vectors, we merge the positive features of each virus, while simultaneously eliminating the safety concerns of each.

The resulting vector has the VSV backbone and, therefore, maintains the rapid replication cycle of wildtype VSV. Furthermore, due to the incorporation of the HN and hyperfusogenic F proteins of NDV, the recombinant virus induces enhanced syncytia formation, allowing for efficient intratumoral spread of the virus and a potent mechanism of tumor cell death and induction of antitumor immune responses. Using this strategy, the benefit of a fusogenic virus can be achieved without the environmental threat associated with NDV.

Additionally, since the endogenous VSV glycoprotein has been deleted, there should be no neurotoxicity associated with the vector. Finally, since NDV attaches to target cells via sialic acid residues, which are upregulated on tumor cells (Bull et al., 2014), we can achieve additional transductional tumor targeting with the pseudotyped vector.

Although numerous pseudotyped VSV vectors have already been reported as safer vectors than wildtype VSV, our specific virus modification differs in that the substitution of the VSV envelope protein with that of NDV results in a more potent virus, in addition to being safer.

Furthermore, we introduce a mutated version of the NDV F protein for further improving the efficacy of the resulting recombinant virus, without negatively impacting safety.

The benefit of this glycoprotein exchange is three-fold:
1. The neurotropism associated with the endogenous VSV glycoprotein can be averted by the deletion of the VSV envelope and the introduction of the non-neurotropic NDV envelope proteins;
2. Tumor cells can be targeted via upregulation of sialic acid residues, which are the natural receptor for NDV; and
3. Viral spread and tumor cell killing can be significantly enhanced via introduction of the highly fusogenic mutant version of the NDV F protein.

Our construct simultaneously provides both improved safety and efficacy.

The pseudotyped virus of the present invention offers improved safety and enhanced efficacy as obvious advantages over wildtype vectors.

Furthermore, there are also advantages of this particular vector over the previously reported pseudotyped VSV vectors. Although the VSV-GP vector (pseudotyped with LCMV-GP) demonstrates an enhanced safety profile, there is no additional therapeutic mechanism afforded by the LCMV glycoprotein in comparison with that of NDV (Muik et al., 2011). Although measles virus (MV) is similar to NDV in that it is a member of the paramyxovirus family, and its envelope also consists of a hemagglutinin and fusion protein, the rVSV-MV vector (Ayala-Breton et al., 2012) does not contain any modification to increase fusigenicity, and it likely is less efficient than our hyperfusogenic VSV-NDV in syncytial formation. Furthermore, MV attaches to target cells via three discrete receptors: CD46, signal lymphocyte activation molecule (SLAM), and nectin4. However, infection of SLAM-positive immune cells leads to immunosuppression, and infection of nectin4-positive airway epithelial cells results in respiratory shedding and virus transmission, both of which would be undesirable side effects of oncolytic virus therapy. Therefore, modifications to ablate the interaction of MV H with SLAM and nectin4 (Liu et al., 2014) or to retarget the attachment protein to tumor specific receptors (Ayala-Breton et al., 2012) has been performed in the context of the rVSV-MV vectors as strategies to retarget the pseudotyped virus to the tumor. However, these restrictions to the natural attachment mechanism of the MV envelope will surely result in an attenuation of the recombinant virus. Indeed, nectin4 and CD46 have substantially overlapping receptor binding surfaces on MV H, and it was shown that disruption of nectin4 binding compromised attachment to CD46, resulting in a greatly diminished oncolytic effect (Liu et al., 2014). Finally, since the majority of the human population is vaccinated against measles virus, the high levels of circulating antibodies directed at the viral envelope will likely play a role in neutralizing the rVSV-MV vectors.

Therefore, our rVSV-NDV vector is superior to the previously reported pseudotyped vectors, due to its hyperfusogenic feature, lack of pre-existing immunity in the general population, and no expected attenuation compared to VSV or NDV.

The following examples and drawings illustrate the present invention without, however, limiting the same thereto.

### BRIEF DESCRIPTION OF THE DRAWINGS

FIG. 1. Recombinant pseudotyped VSV construct expressing the glycoprotein of NDV. The endogenous glycoprotein of VSV was deleted from a plasmid encoding the full-length VSV genome. The NDV glycoprotein, comprising a modified fusion protein (NDV/F (L289A)) and hemagglutinin-neuraminidase (NDV/HN), was inserted as discrete transcription units between the VSV matrix (M) and large polymerase (L) genes. The respective pseudotyped VSV vector was rescued using an established reverse-genetics system.

FIGS. 2A-2D. rVSV-NDV can replicate in HCC cell lines and cause complete cytotoxicity. Human HCC cell lines Huh7 (2A, 2B) and HepG2 (2C, 2D) were infected with a multiplicity of infection (MOI) of 0.01 of rVSV, INDV, or rVSV-NDV. After a 1 hour infection, the cells were washed and fresh medium was added to the cells. At various time-points post-infection aliquots of the supernatant were collected for cytotoxicity measurements by LDH assay (2B, 2D) and cell monolayers were lysed for measurements of intracellular titers by TCID50 assay (2A, 2C). Experiments were performed in triplicate, and data are presented as mean +/−standard deviation.

FIG. 3. rVSV-NDV infection leads to rapid syncytia formation in HCC cells. In order to assess the ability of the pseudotyped rVSV-NDV vector to induce syncytia formation in tumor cells, various HCC cell lines were infected with rVSV-NDV, rNDV, or rVSV at an MOI of 0.01, and observed microscopically at various time-points post-infection. Additional cells were treated with PBS as a control. Huh7 cells are shown as a representative human HCC cell line, and representative images were captured under 200× magnification.

FIG. 4. Pseudotyping VSV with NDV envelope proteins does not alter the sensitivity of the vector to the antiviral actions of IFN. To assess the sensitivity of rVSV-NDV to type I IFN, an IFN-sensitive cell line (A549) was infected with rVSV-NDV, rVSV, and rNDV at an MOI of 0.01. Cells were lysed at 48 hours post-infection, and intracellular viral titers were measured by TCID50 assay. Experiments were performed in triplicate, and mean values +/−standard deviation are shown.

FIGS. 5A-5B. Replication and cytotoxicity of rVSV-NDV is substantially diminished in primary human hepatocytes. Primary human hepatocytes were infected at an MOI of 0.01 with rVSV, rNDV, or rVSV-NDV. Cell lysates were subjected to TCID50 analysis of intracellular virus titers at various timepoints. Additionally, aliquots of supernatant were collected at various timepoints for cytotoxicity measurements by LDH assay. Experiments were performed in duplicate, and means +/−standard deviation are shown.

FIGS. 6A-6B. Replication and cytotoxicity of rVSV-NDV is substantially diminished in primary mouse neurons. Primary mouse neurons were infected at an MOI of 0.01 with rVSV, rNDV, or rVSV-NDV. Cell lysates were subjected to TCID50 analysis of intracellular virus titers at various timepoints. Additional wells were assayed for cell viability using a standard MTS assay. Experiments were performed in duplicate, and means +/−standard deviation are shown.

FIG. 7. The pseudotyped rVSV-NDV vector causes immunogenic cell death. Huh7 cells were infected with rVSV, rNDV, or rVSV-NDV at an MOI of 0.01 or mock-infected for 48 hours. The conditioned media were concentrated, and 10 μg of protein were subjected to Western blot analysis for detection of released HMGB1, Hsp70, and Hsp90.

FIG. 8. Pseudotyped rVSV-NDV vector demonstrates enhanced safety compared to rVSV in immune-deficient mice. Immune-deficient male NOD-SCID mice were treated by tail vein injection with rVSV-NDV or rVSV-GFP (referred to as rVSV in the figure for simplicity) at a dose of 106 TCID50. Mice were monitored daily and euthanized at humane endpoints. Body weight changes were plotted over time with respect to the injection (left); Viral titers in blood were measured on day 1 and 7 by TCID50 analysis (center); The survival proportions were plotted by Kaplan-Maier survival curve (left).

FIG. 9. Mice treated with 106 TCID50 rVSV revealed pathological changes in the liver and brain. H/E staining of liver revealed small group necrosis of hepatocytes after rVSV treatment, marked by hepatocellular degeneration with karyolysis (top left panel). Acute necrosis in the brain stem after rVSV application was observed with degenerating glial cells exhibiting pyknosis and karyorrhexis (top right panel). Degeneration of glial cells could be further confirmed by immunohistochemical staining for caspase-3 (bottom right). Representative images are shown; scale bars equal 50 μm. Viral titers were quantified from brain and liver tissue lysate from mice receiving rVSV after demonstrating signs of toxicity. Means+SEM are shown.

### EXAMPLES

### 1. Material and Methods

### 1.1 Viruses

Recombinant VSV expressing the GFP reporter (referred to herein as “rVSV”) was engineered and rescued as previously described (Huang et al., 2003). Recombinant NDV harboring the F3aa (L289A) mutations and expressing the GFP reporter gene (referred to herein as “rNDV”) was engineered and rescued as previously described (Altomonte et al., 2010).

Recombinant rVSV-NDV was produced by first modifying a plasmid encoding for the full-length VSV genome (pVSV-XN2) and expressing the F3aa (L289A)-modified fusion protein of NDV (Ebert et al., 2004) as an additional transcription unit between the G and L genes. The endogenous VSV glycoprotein (G) was deleted by digestion with MluI and XhoI restriction enzymes, which recognize the unique restriction sites in the 5′ and 3′ noncoding regions of the G, respectively. Following self-ligation of the G-deleted plasmid, a short oligonucleotide linker was inserted at the unique NheI restriction site following the NDV F gene, to create a multiple cloning site for insertion of the HN gene. The HN gene was amplified by PCR from a plasmid encoding the full-length NDV genome, utilizing primers to introduce PacI and PmeI restriction sites at the 5′ and 3′ ends of the PCR product, respectively, for insertion into the newly incorporated restriction sites in the G-deleted VSV-NDV/F3aa (L289A) plasmid. The resulting plasmid was subjected to sequence analysis to confirm the fidelity of the PCR insert, as well as the intergenic transcription start and stop sequences and the gene order. Finally, the infectious virus, referred to here as “rVSV-NDV”, was rescued using the established reverse genetics system for rescuing negative-strand RNA viruses (Lawson et al., 1995).

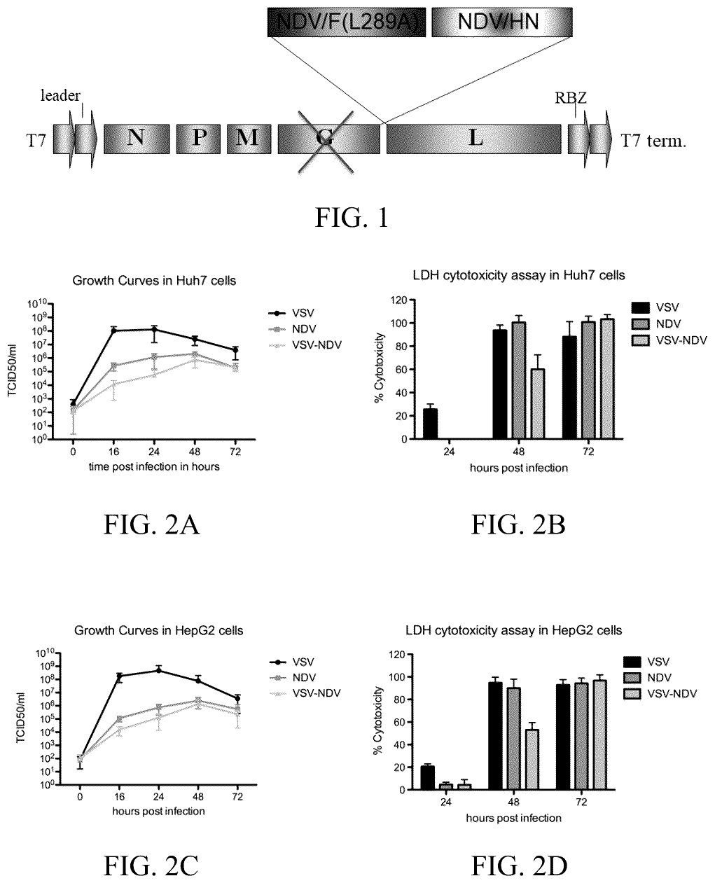

See also FIG. 1.

### 1.2 Cell Lines

Two human HCC cell lines (HepG2 and Huh-7) were obtained from Dr. Ulrich Lauer (University Hospital Tübingen, Germany) and maintained in Dulbecco's modified Eagle's medium (DMEM) supplemented with 10% fetal bovine serum (FBS), 1% L-glutamine (200 mM), 1% Penicillin/streptomycin, 1% non-essential amino acids and 1% sodium pyruvate. A549 cells were obtained from the ATCC (Rockville, MD) and cultured in the same medium as the HCC cell lines. Primary human hepatocytes were derived from patients (negative for hepatitis B and C virus and human immunodeficiency virus) who had undergone surgical resection of liver tumors, in accordance with the guidelines of the charitable state-controlled Human Tissue and Cell Research (HTCR) foundation (Regensburg, Germany). The hepatocytes were maintained in HepatoZYME-SFM medium (Gibco-Invitrogen, Karlsruhe, Germany). Primary embryonic primary cortical neurons were dissociated from E16.5 mouse cortex and provided by the laboratory of Stefan Lichtenthaler (DZNE, Munich, Germany). Neuronal cultures were maintained in Neurobasal medium (Gibco) supplemented with B27 (2%), 0.5 mM glutamine, and 1% penicillin/streptomycin. All cell lines and primary cells were maintained in the 37° C. humidified incubator with 5% CO₂

### 1.3 Microscopic Analysis

The human HCC cell lines, Huh7 and HepG2, were plated at approximately 90% confluency in 6-well dishes and infected with either rVSV, rNDV, or rVSV-NDV at an MOI of 0.01 or mock-infected. Cells were visualized at 200× magnification on an Axiovert 40CFL microscope (Zeiss) at 16-, 24- and 48-hours post-infection, and representative images were captured with a Canon Powershot A620 camera attached to the microscope.

### 1.4 IFN Dose Response Assay

Interferon-sensitive A549 cells were plated in 24-well dishes at a density of 10⁵ cells per well and cultured overnight. The following evening they were pre-treated with different concentrations (0, 100, 500, and 1000 IU/ml) of Universal type I Interferon added directly to the culture medium. After overnight incubation, the cells were infected with either rVSV, rNDV or rVSV-NDV at a multiplicity of infection (MOI) of 0.01. 48 hours post-infection, cells were collected in 100 μl of PBS and lysed by three freeze-thaw cycles. The intratumoral virus titer was determined by TCID₅₀ analysis of the cell lysates.

### 1.5 Growth Curves (TCID50 Assay)

Viral growth curves were performed in HCC cell lines (Huh7 and HepG2), as well as in primary human hepatocytes and primary mouse neurons.

HCC cell lines were plated in 6-well dishes at a density of 3.5×10⁵ cells per well, while PHH and neurons were seeded in collagen-coated 24-well dishes at a density of 10⁵ cells per well. Each cell line was infected with rVSV, rNDV and rVSV-NDV at a multiplicity of infection (MOI) of 0.01. The infections were performed in 1 ml of PBS (6-well dishes) or 250 μl of PBS (24-well dishes) at 37° C. for 1 hour. After incubation, cells were washed three times with PBS and fresh medium was added. Cell lysate was collected at 0, 16, 24, 48 and 72 hours post-infection for TCID₅₀ analysis of intracellular virus titers.

### 1.6 Cytotoxicity Assays (LDH or MTS Assay)

Cell viability of infected HCC cell lines (Huh7 and HepG2) and primary human hepatocytes was analyzed by measuring released Lactate Dehyrogenase (LDH) from cell culture supernatant. The cells were plated, infected and washed as in the growth curve experiments. At 24, 48 and 72 hours post-infection, aliquots of supernatant were collected, and LDH-release was quantified using the CytoTox 96 Non-Radioactive Cytotoxicity Assay protocol (Promega). For each time point, LDH-release following virus infection was calculated as a percentage of the maximum LDH-release control. Baseline LDH levels detected in the supernatant of mock-treated cells were subtracted from the values obtained from the experimental wells.

Cell viability of neurons was analyzed by MTS (3-(4,5-dimethylthiazol-2-yl)-5-(3-carbooxymethoxyphenyl)-2-(4-sulfophenyl)-2H-tetrazolium) assay using the CellTiter96 AQueous One Solution Cell Proliferation Assay (Promega, Madison, WI). Neurons were seeded in collagen coated 96-well dishes at a density of 5×10⁴ cells/well and mock-treated or infected with rVSV, rNDV or rVSV-NDV at an MOI of 0.01. At 24, 48 and 72 hours post-infection, cell viability was measured according to the manufacturer's protocol. Cytotoxicity was calculated as difference in cell viability of the experimental samples compared to the uninfected controls.

### 1.7 Western Blots

Huh7 cells were plated in 6-well plates at approximately 90% confluence and infected with rVSV, rNDV, or rVSV-NDV at an MOI of 0.01 or mock-infected for 48 hours. The conditioned media were collected and concentrated to about 200 μl using Amicon Ultra Centrifugal filters with a 10 kD cutoff (Merck Millipore, Billerica, MA). Protein concentrations were quantified using the Pierce BCA Protein Assay (Thermo Fisher Scientific, Waltham, MA), and 10 μg of each sample was loaded onto a 7.5% denaturing SDS-PAGE gel, followed by transfer onto a nitrocellulose membrane. Protein bands were detected using specific antibodies against HMGB1 and Hsp90 (Cell Signaling Technology, Danvers, MA) and Hsp70 (Santa Cruz Biotechnology, Dallas, TX) and the appropriate secondary antibody conjugated with horseradish peroxidase. Bands were visualized using Amersham ECL Prime Western Blot Detection Reagent (GE Healthcare Life Sciences, Pittsburgh, PA).

### 2. Results

The recombinant VSV-NDV vector (FIG. 1) has been characterized in vitro for replication and cytotoxicity in tumor cells, as well as in healthy hepatocytes and neurons. We used two human hepatocellular carcinoma (HCC) cell lines as representative tumor cells, and compared the rVSV-NDV with rVSV and rNDV in terms of its relative ability to replicate and kill the cells. Although rVSV-NDV replication was a bit delayed compared to the wildtype vectors, it was able to reach similar titers at about 72 hours post-infection, which resulted in complete cell killing in vitro (FIG. 2).

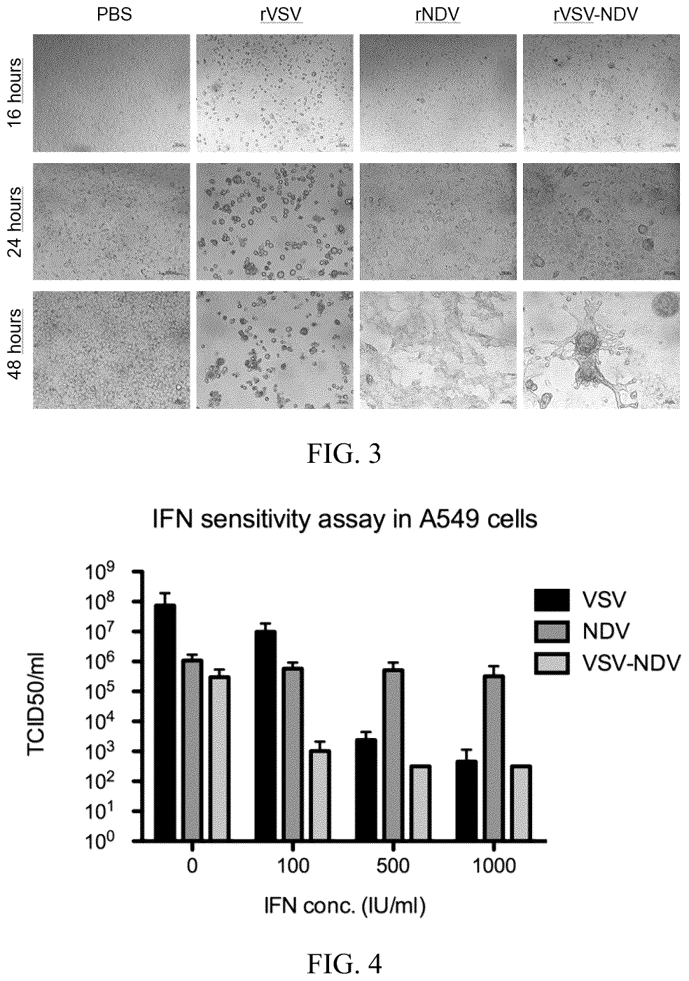

In order to observe virus-induced syncytial formation, additional cells were infected with rVSV-NDV, as well as the parental rVSV and rNDV, for photomicroscopy. Microscopic analysis of the tumor cells revealed multiple foci of syncytia in the wells infected with rVSV-NDV by 16 hours post-infection, while it was significantly delayed in those infected with rNDV. As expected, cells that were treated with rVSV did not form syncytia; however, they were highly susceptible to the cytopathic effect (CPE), which is classic of VSV infection and occurred earlier than 16 hours post-infection (FIG. 3).

In order to rule out that the glycoprotein exchange inadvertently resulted in a loss of sensitivity of the vector to the antiviral actions of type I interferon (IFN), an IFN dose response was performed. The exquisite sensitivity of VSV to type I IFN is a key mechanism of tumor specificity, as tumor cells are often defective in their IFN signaling pathways, while healthy cells can efficiently clear the virus via IFN responsive genes. Although this assay revealed a relative insensitivity of rNDV to type I IFN, the rVSV-NDV vector was rapidly attenuated by the addition of IFN and reduced to levels similar to those observed for rVSV (FIG. 4).

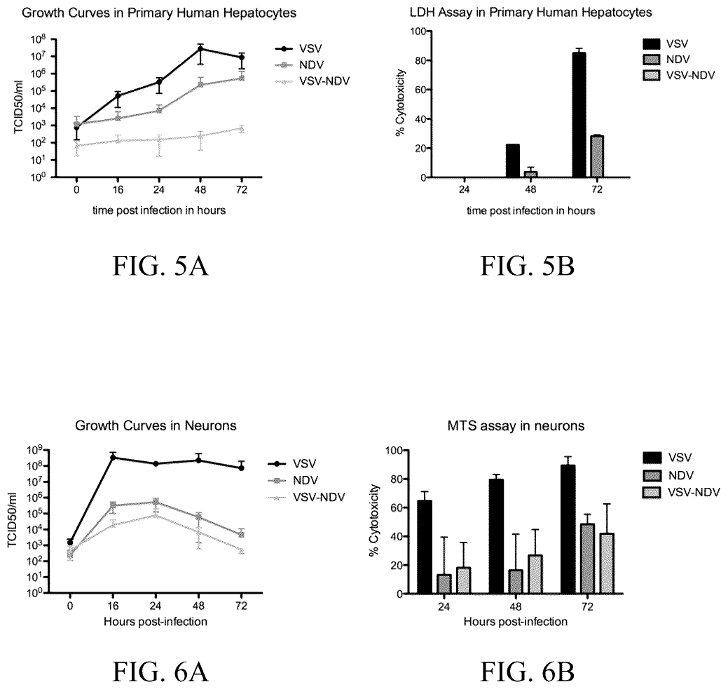

We next performed growth curves and cytotoxicity assays in normal primary human hepatocytes and mouse neurons in order to assess the safety of rVSV-NDV. Very little replication of the pseudotyped vector could be observed over time, and titers were approximately 5 logs lower than the control VSV vector at 48 hours post-infection and 3 logs lower than rNDV at the same time-point in primary hepatocytes (FIG. 5). Although nearly all hepatocytes were dead by 72 hours post-infection with rVSV, no cytotoxicity could be observed by LDH assay in cells infected with rVSV-NDV (FIG. 5). Similarly, titers of rVSV-NDV were significantly lower than the control VSV vector in primary mouse neurons at all time-points investigated, which corresponded to similar levels of cell viability as those observed in PBS-treat neurons (FIG. 6). Taken together, rVSV-NDV showed little evidence of replication in primary healthy cells and resulted in little to no cytotoxicity in vitro, indicating that it is a substantially safer virus than both rVSV and rNDV.

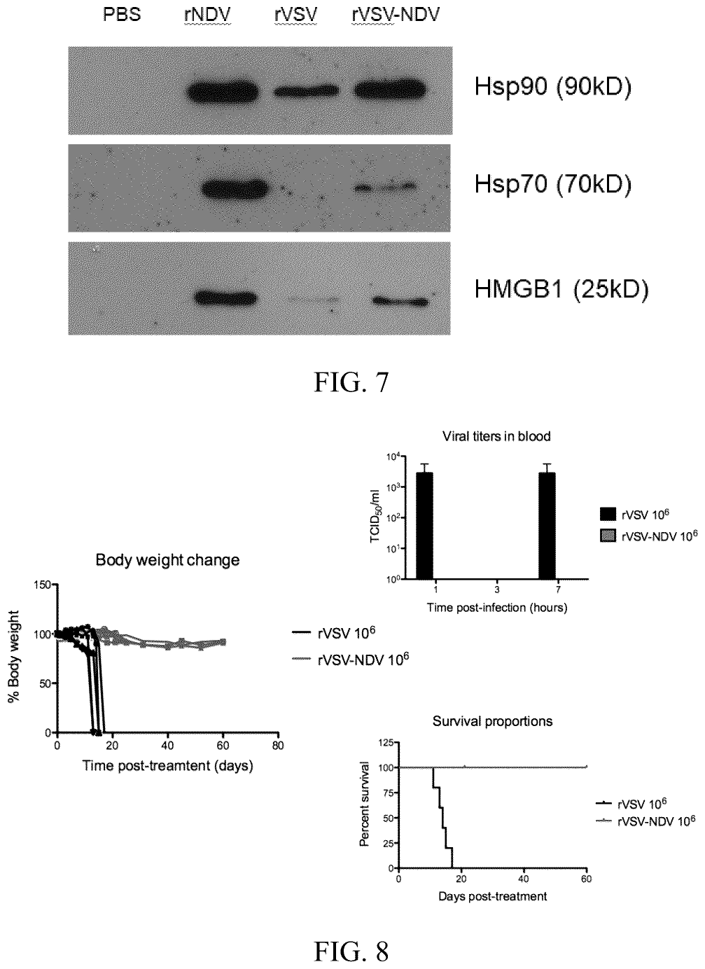

To determine whether the pseudotyped rVSV vector would induce an immunogenic cell death, as has been shown for rNDV through syncytia formation, we investigated the release of high mobility group box 1 (HMGB1) and heat-shock proteins 70 and 90 from infected Huh7 cells. After a 48 hour infection, we observed relatively low levels of HMGB1, Hsp70, and Hsp90 released into the supernatant of rVSV-infected cells. However, infection with both rNDV and rVSV-NDV resulted in high levels of all three secreted markers for immunogenic cell death (FIG. 7). These results indicate that, in addition to the potent direct cytotoxicity caused by infection with the pseudotyped rVSV-NDV vector, in vivo treatment with this virus could result in substantial immune responses directed against the tumor.

In order to assess the safety of the pseudotyped rVSV-NDV vector in vivo, immune-deficient male NOD-SCID mice approximately 8 weeks of age were treated by tail vein injection with either rVSV-NDV or the control rVSV-GFP virus (N=6) at a dose of 10⁶ TCID50 per mouse. Mice were monitored daily for body weight and overall physical appearance, and they were euthanized at humane endpoints. Blood was sampled on day 1, 3, 7, 14, and at the time of euthanization for analysis serum chemistry and circulating virus titers. Two mice receiving rVSV-GFP rapidly began losing weight during the first week after treatment, and all six died acutely or were euthanized due to extreme body weight loss, dehydration, signs of distress (changes in posture, impaired movement, isolation, etc.), and/or signs of neurotoxicity (limb paralysis and circling) between 11 and 17 days post-treatment (FIG. 8). Additionally, infectious virus titers could be recovered from the blood on day 1 and 7 post-treatment (FIG. 8, center). In contrast, the mice who received rVSV-NDV lost only negligible amounts of weight, appeared healthy and exhibited normal behavior throughout the study. Three of these mice were euthanized at 21 days post-treatment for histological analysis of major organs, while the remaining animals were monitored for 60 days post-treatment, at which time they were euthanized for pathological analysis. No infectious virus titers could be detected in the blood of mice treated with rVSV-NDV at any time-point analyzed. Plasma measurements of liver function (GPT) and kidney function (BUN and Creatinine) revealed no abnormal values for either treatment group (data not shown).

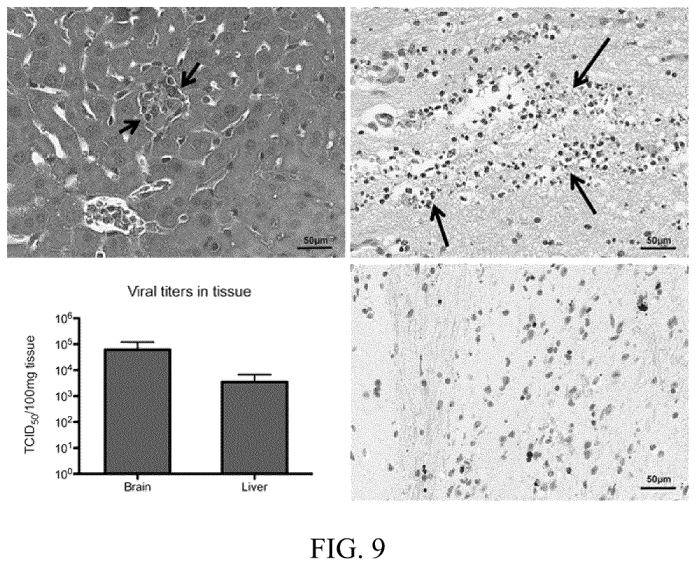

Tissue sections were examined by a pathologist who was blinded to the treatment groups of the specimens. Histological analysis revealed no major pathological findings in tissue excised from mice treated with rVSV-NDV, either euthanized on day 21 or day 60. Furthermore, no detectable titers within the brain or liver tissue could be observed in mice treated with rVSV-NDV (data not shown). In stark contrast, mice that received rVSV-GFP at the same dose exhibited heavy intrasinusoidal edema, moderate acute hepatitis with single cell and small group necrosis, and apoptosis of hepatic tissue (FIG. 9). Furthermore, acute necrosis in the brain stem, with degenerating glial cells exhibiting pyknosis and karyorhexis could be observed. Degeneration of glial cells was further confirmed by immunohistochemical staining for caspase-3. TCID50 analysis of tissue lysates revealed quantifiable levels of infectious VSV in the liver and brain at the time of necropsy.

The features disclosed in the foregoing description, in the claims and/or in the accompanying drawings may, both separately and in any combination thereof, be material for realizing the invention in diverse forms thereof.

### REFERENCES

Altomonte, J., L. Wu, et al. (2008). “Exponential enhancement of oncolytic vesicular stomatitis virus potency by vector-mediated suppression of inflammatory responses in vivo.” Mol Ther 16 (1): 146-153.
Altomonte, J., S. Marozin, et al. (2010). “Engineered newcastle disease virus as an improved oncolytic agent against hepatocellular carcinoma.” Mol Ther 18 (2): 275-284.
Ayala-Breton, C., G. N. Barber, et al. (2012). “Retargeting vesicular stomatitis virus using measles virus envelope glycoproteins.” Hum Gene Ther 23 (5): 484-491.
Bull, C., M. A. Stoel, et al. (2014). “Sialic acids sweeten a tumor's life.” Cancer Res 74 (12): 3199-3204.
Coffey, M. C., Strong, J. E., Forsyth, P. A., Lee, P. W. (1998). “Reovirus therapy of tumors with activated Ras pathway.” Science 282:1332-1334.
Cuadrado-Castano, S., M. T. Sanchez-Aparicio, et al. (2015). “The therapeutic effect of death: Newcastle disease virus and its antitumor potential.” Virus Res 209:56-66.
Ebert, O., K. Shinozaki, et al. (2004). “Syncytia induction enhances the oncolytic potential of vesicular stomatitis virus in virotherapy for cancer.” Cancer Res 64 (9): 3265-70.
Ebert, O., S. Harbaran, et al. (2005). “Systemic therapy of experimental breast cancer metastases by mutant vesicular stomatitis virus in immune-competent mice.” Cancer Gene Ther 12 (4): 350-358.
Edge, R. E., T. J. Falls, et al. (2008). “A let-7 MicroRNA-sensitive vesicular stomatitis virus demonstrates tumor-specific replication.” Mol Ther 16 (8): 1437-1443.
Everts, B. and H. G. van der Poel (2005). “Replication-selective oncolytic viruses in the treatment of cancer.” Cancer Gene Ther 12 (2): 141-161.
Huang H. G., O. Ebert, et al. (2003). “Oncolysis of hepatic metastasis of colorectal cancer by recombinant vesicular stomatitis virus in immune competent mice.” Mol Ther 8 (3): 434-40.
Johnson, J. E., F. Nasar, et al. (2007). “Neurovirulence properties of recombinant vesicular stomatitis virus vectors in non-human primates.” Virology 360 (1): 36-49.
Kelly, E. J., R. Nace, et al. (2010). “Attenuation of vesicular stomatitis virus encephalitis through microRNA targeting.” J Virol 84 (3): 1550-1562.
Kirn, D., Martuza, R. L., and Zwiebel, J. (2001). “Replication-selective virotherapy for cancer: biological principles, risk management, and future directions.” Nat Med 7:781-787.
Lawson, N. D., E. A. Stillman, et al. (1995). “Recombinant vesicular stomatitis viruses from DNA.” Proc Natl Acad Sci 92:4477-81.
Liu, Y. P., S. P. Russell, et al. (2014). “Ablation of nectin4 binding compromises CD46 usage by a hybrid vesicular stomatitis virus/measles virus.” J Virol 88 (4): 2195-2204.
Lorence, R. M., Katubig, B. B., Reichard, K. W., et al (1994). “Complete regression of human fibrosarcoma xenografts after local Newcastle disease virus therapy.” Cancer Research 54:6017-6021.
Muik, A., I. Kneiske, et al. (2011). “Pseudotyping vesicular stomatitis virus with lymphocytic choriomeningitis virus glycoproteins enhances infectivity for glioma cells and minimizes neurotropism.” J Virol 85 (11): 5679-5684.
Patel, M. R. and R. A. Kratzke (2013). “Oncolytic virus therapy for cancer: the first wave of translational clinical trials.” Transl Res 161 (4): 355-364.
Park, M. S., Steel, J., Garcia-Sastre, A., et al (2006). “Engineered viral vaccine constructs with dual specificity: Avian influenza and Newcastle disease.” PNAS 103 (21): 8203-8.
Peeters, B. P., O. S. de Leeuw, et al. (1999). “Rescue of Newcastle disease virus from cloned cDNA: evidence that cleavability of the fusion protein is a major determinent of virulence.” J Virol 73 (6): 5001-9.
Peng, K. W., Ahmann, G. J., Pham, L, et al (2001). “Systemic therapy of myeloma xenografts by an attenuated measles virus.” Blood 98:2002-2007.
Quiroz, E., N. Moreno, et al. (1988). “A human case of encephalitis associated with vesicular stomatitis virus (Indiana serotype) infection.” Am J Trop Med Hyg 39 (3): 312-314.
Stojdl, D. F., Lichty, B. D., tenOever, B. R., et al (2003). “VSV strains with defects in their ability to shutdown innate immunity are potent systemic anti-cancer agents.” Cancer Cell 4 (4): 263-275.
van den Pol, A., Dalton, K., and Rose, J. (2002). “Relative neurotropism of a recombinant rhabdovirus expressing a green fluorescent envelope glycoprotein.” Journal of Virology 76 (3): 1309-1327.
Vigil, A., M. S. Park, et al. (2007). “Use of reverse genetics to enhance the oncolytic properties of Newcastle disease virus.” Cancer Res 67 (17): 8285-8292.

1. A method for producing a recombinant oncolytic virus, comprising a vesicular stomatitis virus (VSV), wherein the method comprises the steps of deleting glycoprotein (G protein) of VSV, and wherein the VSV comprises a modified fusion protein (F protein) of Newcastle disease virus (NDV), and the hemagglutinin neuraminidase (HN) protein of NDV.

2. The method of claim 1, wherein the modified fusion protein (F protein) of NDV is a F3aa-modified F protein,
and/or
wherein the modified fusion protein comprises at least one amino acid substitution in the protease cleavage site,
and/or
wherein the G protein of VSV is replaced by the modified fusion protein and the HN protein of NDV.

3. The method of claim 1, wherein the modified fusion protein (F protein) of NDV comprises an amino acid sequence having at least 95% sequence identity to the amino acid sequence of SEQ ID NOs: 10 or 12,
and/or
wherein the HN protein of NDV comprises an amino acid sequence having at least 95% sequence identity to the amino acid sequence of SEQ ID NO: 6.

4. A recombinant oncolytic virus, comprising a vesicular stomatitis virus (VSV), wherein the glycoprotein (G protein) of VSV is deleted, and which comprises a modified fusion protein (F protein) of Newcastle disease virus (NDV); and the hemagglutinin neuraminidase (HN) protein of NDV,
wherein the modified fusion protein (F protein) of NDV comprises an amino acid sequence having at least 95% sequence identity to the amino acid sequence of SEQ ID NOs: 10 or 12, and/or
wherein the HN protein of NDV comprises an amino acid sequence having at least 95% sequence identity to the amino acid sequence of SEQ ID NO: 6.

5. A nucleic acid encoding a recombinant oncolytic virus according to claim 4.

6. A vector comprising a nucleic acid of claim 5.

7. A pharmaceutical composition, comprising:
(a) (i) a recombinant oncolytic virus produced by a method for producing a recombinant oncolytic virus, comprising a vesicular stomatitis virus (VSV), wherein the method comprises the steps of deleting glycoprotein (G protein) of VSV, and wherein the VSV comprises a modified fusion protein (F protein) of Newcastle disease virus (NDV), and the hemagglutinin neuraminidase (HN) protein of NDV; or
(ii) a recombinant oncolytic virus, comprising a vesicular stomatitis virus (VSV), wherein the glycoprotein (G protein) of VSV is deleted, and which comprises a modified fusion protein (F protein) of Newcastle disease virus (NDV); and the hemagglutinin neuraminidase (HN) protein of NDV,
wherein the modified fusion protein (F protein) of NDV comprises an amino acid sequence having at least 95% sequence identity to the amino acid sequence of SEQ ID NOs: 10 or 12,
and/or
wherein the HN protein of NDV comprises an amino acid sequence having at least 95% sequence identity to the amino acid sequence of SEQ ID NO: 6; or
(iii) a nucleic acid encoding a recombinant oncolytic virus according to claim 5; and
(b) pharmaceutically acceptable carrier(s) and/or excipient(s).

8. The pharmaceutical composition of claim 7, further comprising one or more compounds selected from
chemotherapeutic agents,
radiotherapeutic agents,
tumor vaccines,
immune checkpoint inhibitors,
cell carrier systems,
small molecule inhibitors,
embolization agents, and
shielding polymers.

9. The pharmaceutical composition of claim 7, formulated for delivery via a route selected from intravenous administration or intra-arterial administration, intradermal, subcutaneous, intramuscular, intravenous, intratumoral, intraosseous, intraperitoneal, intrathecal, epidural, intracardiac, intraarticular, intracavemous, intracerebral, intracerebroventricular and intravitreal injection.

10. A method for oncolytic therapy wherein said method comprises the step of administering to a patient a therapeutically effective amount of:
a recombinant oncolytic virus produced by a method for producing a recombinant oncolytic virus, comprising a vesicular stomatitis virus (VSV), wherein the method comprises the steps of deleting glycoprotein (G protein) of VSV, and wherein the VSV comprises a modified fusion protein (F protein) of Newcastle disease virus (NDV), and the hemagglutinin neuraminidase (HN) protein of NDV; or
a nucleic acid encoding the recombinant oncolytic virus according to claim 5.

11. The method according to claim 10, further comprising the step of administering one or more additional agents selected from cell carrier systems, immunotherapies, and standard tumor therapies, to said patient.

12. A method of treatment of cancer comprising the step of administering to a subject in need thereof a therapeutically effective amount of:
a recombinant oncolytic virus produced by a method for producing a recombinant oncolytic virus, comprising a vesicular stomatitis virus (VSV), wherein the method comprises the steps of deleting glycoprotein (G protein) of VSV, and wherein the VSV comprises a modified fusion protein (F protein) of Newcastle disease virus (NDV), and the hemagglutinin neuraminidase (HN) protein of NDV; or
a nucleic acid encoding the recombinant oncolytic virus according to claim 5.

13. The method according to claim 12, wherein the cell carrier system is selected from T cells, dendritic cells, NK cells, and mesenchymal stem cells; the immunotherapy is selected from tumor vaccines and immune checkpoint inhibitors; and the standard tumor therapy is selected from radiofrequency ablation, chemotherapy, embolization, and small molecule inhibitors.

14. The vector according to claim 6, further comprising one or more reporter genes and/or genes to be delivered to a target cell or tissue.

15. The vector according to claim 14, wherein the reporter gene is selected from HSV1-sr39TK, the sodium iodide symporter (NIS), somatostatin receptor 2 (SSTR2), luciferase, green fluorescence protein (GFP), lacZ, and tyrosinase; and the gene to be delivered to a target cell or tissue is selected from immune stimulating genes, immune checkpoint inhibitory antibodies, and tumor associated antigens (TAA).

16. A method of delivering genes, of imaging of virus biodistribution and/or for tumor detection, wherein the vector of claim 14 is administered to a patient.


SEQUENCE LISTING (17 sequences)

SEQ ID NO: 1 - DNA, 11161 bp, Vesicular stomatitis Indiana virus
  source 1..11161: mol_type = genomic DNA
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 2 - DNA, 1665 bp, Vesicular stomatitis Indiana virus
  source 1..1665: mol_type = genomic DNA
aacagagatcgatctgtttccttgacaccatgaagtgctttttgtacttagcttttttattcatcggggtgaattgcaagttcaccatagtttttccacacaaccaaaaaggaaactggaaaaatgttccttccaattaccattattgcccgtcaagctcagatttaaattggcataatgacttaataggcacaggcttacaagtcaaaatgcccaagagtcacaaggctattcaagcagacggttggatgtgtcatgcttccaaatgggtcactacttgtgatttccgctggtacggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgcttgttgatgaatacacaggagaatgggttgattcacagttcatcaacggaaaatgcagcaatgacatatgccccactgtccataactccacaacctggcattccgactataaggtcaaagggctatgtgattctaacctcatttccacggacatcaccttcttctcagaggacagagagctatcatccctaggaaaggagggcacagggttcagaagtaactactttgcttatgaaactggagacaaggcctgcaaaatgcagtactgcaagcattggggagtcagactcccatcaggtgtctggttcgagatggctgataaggatctctttgctgcagccagattccctgaatgcccagaagggtcaagtatctctgctccatctcagacctcagtggatgtaagtctcattcaggacgttgagaggatcttggattattccctctgccaagaaacctggagcaaaatcagagcgggtcttcccatctctccagtggatctcagctatcttgctcctaaaaacccaggaaccggtcctgcctttaccataatcaatggtaccctaaaatactttgagaccagatacatcagagtcgatattgctgctccaatcctctcaagaatggtcggaatgatcagtggaactaccacagaaagggaactgtgggatgactgggctccatatgaagacgtggaaattggacccaatggagttctgaggaccagttcaggatataagtttcctttatatatgattggacatggtatgttggactccggtcttcatcttagctcaaaggctcaggtgtttgaacatcctcacattcaagacgctgcttcgcagcttcctgatgatgagattttattttttggtgatactgggctatccaaaaatccaatcgactttgtcgaaggttggttcagtagttggaagagctccattgcctcttttttctttatcatagggttaatcattggactattcttggttctccgagttggtatttatctttacattaaattaaagcacaccaagaaaagacagatttatacagacatagagatgaaccgacttggaaggtaactcaaatcctgcacaacagattcttcatgtttggaccaaatcaacttgtgataccatgctcaaagaggcctcaattatatttgagtttttaatttttatg

SEQ ID NO: 3 - protein, 511 aa, Vesicular stomatitis Indiana virus
MKCFLYLAFLFIGVNCKFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 4 - DNA, 15186 bp, Newcastle disease virus
  source 1..15186: mol_type = genomic DNA
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 5 - DNA, 1992 bp, Newcastle disease virus
  source 1..1992: mol_type = genomic DNA
acgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgat

SEQ ID NO: 6 - protein, 577 aa, Newcastle disease virus
MDRAVSQVALENDEREAKNTWRLIFRIAILFLTVVTLAISVASLLYSMGASTPSDLVGIPTRISRAEEKITSTLGSNQDVVDRIYKQVALESPLALLNTETTIMNAITSLSYQINGAANNSGWGAPIHDPDYIGGIGKELIVDDASDVTSFYPSAFQEHLNFIPAPTTGSGCTRIPSFDMSATHYCYTHNVILSGCRDHSHSHQYLALGVLRTSATGRVFFSTLRSINLDDTQNRKSCSVSATPLGCDMLCSKATETEEEDYNSAVPTRMVHGRLGFDGQYHEKDLDVTTLFGDWVANYPGVGGGSFIDSRVWFSVYGGLKPNTPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSDKTATLHSPYTFNAFTRPGSIPCQASARCPNSCVTGVYTDPYPLIFYRNHTLRGVFGTMLDGEQARLNPASAVFDSTSRSRITRVSSSSIKAAYTTSTCFKVVKTNKTYCLSIAEISNTLFGEFRIVPLLVEILKDDGVREARSG

SEQ ID NO: 7 - DNA, 1782 bp, Newcastle disease virus
  source 1..1782: mol_type = genomic DNA
acgggtagaagactctggatcccggttggcgccctccaggtgcaggatgggctccagaccttttaccaagaacccagcacctatgatgctgactatccgggtcgcgctggtattgagttgcatctgtccggcaaactccattgatggcaggccttttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcccaactagcagtggcagttgggaagatgcagcagtttgttaatgaccaatttaataaaacagctcaggaattagactgcataaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagt

SEQ ID NO: 8 - protein, 553 aa, Newcastle disease virus
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 9 - DNA, 1662 bp, synthetic construct
  misc_feature 1..1662: note = NDV F3aa-modified fusion protein
  source 1..1662: mol_type = other DNA
atgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 10 - protein, 553 aa, synthetic construct
  REGION 1..553: note = NDV F3aa-modified fusion protein
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 11 - DNA, 1662 bp, synthetic construct
  misc_feature 1..1662: note = NDV F3aa-modified fusion protein with L289A
  source 1..1662: mol_type = other DNA
atgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 12 - protein, 553 aa, synthetic construct
  REGION 1..553: note = NDV F3aa-modified fusion protein with L289A
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTAPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 13 - DNA, 13006 bp, synthetic construct
  misc_feature 1..13006: note = Virus/vector construct
  source 1..13006: mol_type = other DNA
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcgagatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgagctagcttaattaatatgaaaaaaactaacagatatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctaggtttaaacgctagcggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 14 - protein, 422 aa, Vesicular stomatitis Indiana virus
MSVTVKRIIDNTVIVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 15 - protein, 265 aa, Vesicular stomatitis Indiana virus
MDNLTKVREYLKSYSRLDQAVGEIDEIEAQRAEKSNYELFQEDGVEEHTKPSYFQAADDSDTESEPEIEDNQGLYAQDPEAEQVEGFIQGPLDDYADEEVDVVFTSDWKPPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 16 - protein, 229 aa, Vesicular stomatitis Indiana virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 17 - protein, 2109 aa, Vesicular stomatitis Indiana virus
MEVHDFETDEFNDFNEDDYATREFLNPDERMTYLNHADYNLNSPLISDDIDNLIRKFNSLPIPSMWDSKNWDGVLEMLTSCQANPISTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWTILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRTSDSLAPIGNWIRSLELVRNQVRLNPFNEILFNQLCRTVDNHLKWSNLRRNTGMIEWINRRISKEDRSILMLKSDLHEENSWRD